(19) United States
(12) Patent Application Publication
(10) Pub. No.: US 2015/0250945 A1
(43) Pub. Date: Sep. 10, 2015

(54) CONNECTOR TYPE FILTER FOR SYRINGE, SYRINGE WITH A FILTER AND MANUFACTURING METHOD THEREOF

(71) Applicant: IMTKORE A CO., LTD., Gyeonggi-do (KR)

(72) Inventor: Jong-Uk Kim, Daegu (KR)

(21) Appl. No.: 14/427,310

(22) PCT Filed: Sep. 10, 2013
(86) PCT No.: PCT/KR2013/008141

§ 371 Date: Mar. 11, 2015

(30) Foreign Application Priority Data
Jul. 23, 2013 (KR) 10-2013-0086785

(51) Int. Cl. A61M 5/165; A61M 5/34

(52) U.S. Cl. CPC A61M 5/165; A61M 5/34

(57) ABSTRACT

Disclosed is a syringe with a connector-type filtering device for use in administering a medicinal fluid to a patient, including: a cylinder having a space formed to store the medicinal fluid therein; a piston configured to reciprocate within the space of the cylinder to suck in or discharge the medicinal fluid; a connector member assembled/dissembled between a needle holder and a neck a in front of the cylinder and having a function for removing foreign matter from the medicinal fluid; the needle holder configured such that the connector member is fitted in or disassembled from the needle holder; and a needle insert-molded on a tip end of the needle holder.

### TECHNICAL FIELD

The present invention relates to a connector-type filtering device for a syringe. More particularly, the present invention relates to a connector-type filtering device for a syringe configured in the form of a connector which is an intermediate medium capable of being interposed between a needle holder and a cylinder of the syringe, in which the filtering device may be applied for both suction and discharge operations of a medicinal fluid so as to make the syringe compatible with a commercially available conventional syringe so that the syringe can be easily and conveniently used, and to maximize safety at the time of injecting the medicinal fluid by dual filtration, including primary filtration at the time of sucking in the medicinal fluid and secondary filtration at the time of discharging the medicinal fluid. The present invention also relates to a syringe provided with the connector-type filtering device and a method for manufacturing the connector-type filtering device.

### BACKGROUND ART

In general, a syringe is used for directly injecting a medicinal fluid to a patient, in which the medicinal fluid is prepared by mixing a liquid phase and powder or only by a liquid phase. In order to prevent secondary infection during the process of injection, a disposable syringe is usually used. It is necessary to replace the needle and to administer a medicinal liquid using a new needle due to the patient's narrow blood vessel or the high viscosity of the medicinal fluid. Or, it may be necessary to directly administer the medicinal fluid to an injection port. Thus, in order to allow the needle holder to be separated from the cylinder of the syringe to replace the needle holder or, in a state where the needle holder is removed, to use the cylinder by directly connecting the cylinder to the injection port, as needed, the needle holder and the cylinder of the syringe are configured to be separable from each other and formed according to ISO international standards.

As exemplified in FIG. 1, an ordinary syringe includes: a cylinder 1 defining a space so as to store a medicinal fluid therein; a piston 2 configured to reciprocate within the space of the cylinder 1 so as to suck in or discharge the medicinal fluid; a needle holder 3 fitted on an outer circumferential surface of a neck 1a formed in front of the cylinder 1; and a needle 4 insert-molded on a tip end of the needle holder 3.

Meanwhile, a liquid phase of a medicinal fluid for use in the syringe is contained in an ampule usually made of glass or plastic. Thus, when the ampule is opened, glass powder or powder of other foreign matter including plastic, may be generated and mixed in the medicinal fluid. Such foreign matter may be sucked in to the inside of the cylinder of the syringe, together with the medicinal fluid, and injected into a human body as it is without being filtered. It is known that such foreign matter may cause a lot of side effects.

That is, most of ampules are made of glass in order to prevent pollution of the ampule during the storage and transportation thereof. Such an ampule is partly broken to form an outlet, or a needle is forcibly inserted into a plug closing the ampule, which is formed of rubber, so as to suck in the medicinal fluid to the inside of the cylinder of a syringe. During this process, fine glass powder or rubber pieces may be mixed in the medicinal fluid, introduced into the syringe, and then administered to a patient, together with the medicinal fluid.

Accordingly, in the medical fields, in order to solve the problems described above, it is practically recommended, when sucking and charging the medicinal fluid into the syringe, using a needle equipped with a filter and a holder and, when injecting the medicinal fluid to the patient after the medicinal fluid is charged in the syringe, replacing the needle and the holder with new ones and then injecting the medicinal fluid to the patient.

However, a series of the operations described above are actually very inconvenient and make it difficult to quickly cope with an emergency patient. In addition, as multiple needles and holders are used for every injection, a lot of disposable syringes are used in one day and medical waste increases geometrically, which may cause subsidiary problems of, for example, exerting adverse effects on environment.

As a prior art technique developed in order to solve the problems described above, Korean Patent No. 10-1245378 (issued on Mar. 19, 2013) (hereinafter, simply referred to as “Patent Document 1”) discloses a technique related to a filter needle, in which a filter is incorporated in a section of a syringe that contains a medicinal fluid to be administered to a patient to be sucked in and discharged so as to prevent foreign matter from being contained in the medicinal fluid at the time of charging the medicinal fluid from the ampule to the inside of the syringe, and at the time of administering the medicinal fluid to the patient. FIG. 2 exemplifies a schematic view of the filter needle.

According to the filter needle disclosed in Patent Document 1, a filter structure constituted with a circular body 10 and a filter 20 is incorporated in a holder 3 that fixes a needle 4 to the syringe. The filter structure is configured to separate an inlet 5a and an outlet 5b from one another by the circular body including a screen membrane 12, a cutoff line 12a, and an edge membrane 11, while allowing each of the inlet 5a and the outlet 5b to be opened in one way only by a check valve structure. A filter 20 is mounted on the outlet 5b to prevent the discharge of foreign matter, thereby preventing the foreign matter remaining in the inside of the syringe from being administered to the patient. In addition, since the structure of the filter 20 is formed in a shape of a cylindrical pipe, which includes a cylindrical membrane 21 and a blocking membrane 22, and is mounted to be exposed to the back of the valve, a cross-sectional area for filtering the medicinal fluid increases, thereby distributing the filtering pressure at the time of discharging the medicinal fluid so that the pressure applied to the piston may be reduced and the injection of the medicinal fluid to the patient may be facilitated.

In the prior art disclosed in Patent Document 1, however the needle and the filter are formed integrally by artificially deforming the needle to increase the length of the needle and then mounting the filter structure inside the needle holder, and as a result, are not compatible with an ordinary syringe. Thus, the filtering function cannot be used in a case where, due to a kind of medicinal fluid or a size of a blood vessel, the needle is replaced with another needle of a different gauge in order to administer a medicinal fluid, or the cylinder of the syringe is directly connected to an injection port in a state where the needle is removed so as to administer the medicinal fluid.

Meanwhile, U.S. Pat. No. 4,332,249 (issued on Jun. 1, 1982 and entitled “Filter and Valve Assembly for hypodermic Syringe) (hereinafter, simply referred to as “Patent Document 2”) discloses a technique related to a filter and valve assembly for a hypodermic syringe. FIGS. 3a and 3b exemplify cross-sectional views of a principal part thereof.

According to the filter and valve assembly for the hypodermic syringe disclosed in Patent Document 2, a filtering device 30, including a filter paper 31 and an elastic valve 32, is attached to the inside of a needle holder 3 so as to configure a needle 4 and the filtering device 30 integrally. The structure of the elastic valve 32 only allows one way opening so as to form a medicinal fluid inlet from the needle toward the inside of the cylinder, and a medicinal fluid outlet is formed from the inside of the cylinder toward the needle by the filter paper 31 so that foreign matter may be filtered through the filter paper.

The structure of Patent Document 2 is not compatible with an ordinary syringe like that of Patent Document 1, and thus, should only use a designated syringe. Since the filter paper used for the filtering device is made of a very thick filter paper 31, the fluid pressure is not smoothly formed at the time of sucking in or discharging the medicinal fluid, and thus, there is a lot of inconvenience in use.

In addition, Korean Patent No. 10-1165946 (issued on Jul. 10, 2012) (hereinafter, simply referred to as “Patent Document 3”) discloses a technique related to a filter member for a syringe which is configured such that, when the medicinal fluid is sucked in to the inside of the syringe, the medicinal fluid does not pass through a filter, and as a result, the medicinal fluid may be smoothly sucked in. Whereas, when the medicinal fluid sucked in to the inside of the syringe is administered to a human body, the medicinal fluid sucked in to the inside of the syringe passes through the filter so that the foreign matter included in the medicinal fluid can be filtered. FIG. 4 exemplifies a schematic view of Patent Document 3.

According to Patent Document 3, the filter member disclosed therein includes: a needle holder 41 coupled to a needle 4 at the front side and opened at the rear side, and having a first installation portion 41a and a second installation portion 41b provided in succession toward the rear side; a middle block 42 incorporated in the first installation portion 41a and having a suction through-hole 42a formed to penetrate the middle block 42 in an axial direction so as to allow medicinal fluid sucked in to the suction through-hole 42a to pass therethrough; an end cap 43 rotatably inserted into the second installation portion 41b at the front side thereof and opened to be fitted on a cylinder neck 1a of a cylinder 1 at the rear side thereof, the end cap 43 including, at the front side, a filter hole 43a and a communication hole 43b, which are formed to selectively communication with the suction through-hole 42a; and a filter 44 configured to close the filter hole of the end cap.

Due to the complicated filtering structure of Patent Document 3, however, the needle holder becomes fat, and thus, it is necessary to use only a dedicated syringe rather than an ordinary conventional syringe.

In addition, Patent Documents 1 to 3 are configured such that all the medicinal fluid and foreign matter flow into the inside of the cylinder of a syringe without passing through the filter, and foreign matter is filtered at the time of discharging the medicinal fluid, or foreign matter is filtered at the time of introducing the medicinal fluid into the inside of the cylinder of the syringe, but is not filtered at the time of discharging the medicinal fluid. Thus, the prior arts disclosed in Patent Documents 1 to 3 only implement a primary filtration function in which the syringe cannot be used by being directly coupled to an injection port. Thus, there is a problem in that the foreign matter cannot be safely removed when the medicinal fluid is injected to the patient.

### PRIOR ART DOCUMENT

### Patent Document

(Patent Document 1) KR Patent No. 10-1245378 B1 issued on Mar. 19, 2013

(Patent Document 2) U.S. Pat. No. 4,332,249 issued on Jun. 1, 1982

(Patent Document 3) KR Patent No. 10-1165946 B1 issued on Jul. 10, 2012

### DETAILED DESCRIPTION OF THE INVENTION

### Technical Problem

Accordingly, the present invention was made in an effort to solve the above-described problems of the prior art and the present invention is to provide a connector-type filtering device for a syringe which is configured in the form of a connector as an intermediate medium which is capable of being interposed between a needle holder and a cylinder of the syringe, to be compatible with a conventional syringe, and is applied for each of suction and discharge operations of a medicinal fluid so that both filtration at the time of suction of the medicinal fluid and filtration at the time of discharge of the medicinal fluid are enabled, thereby improving safety. The present invention is also to provide a syringe including the connector-type filtering device, and a method for manufacturing the connector-type filtering device.

### Technical Solution

In order to achieve the above-described object, the present invention provides a syringe for administering a medicinal fluid to a patient. The syringe includes: a cylinder defining a space for storing a medicinal fluid therein; a piston configured to reciprocate within the space of the cylinder so as to suck in or discharge a medicinal fluid; a connector member assembled/dissembled between a needle holder and a neck in front of the cylinder and having a function for removing foreign matter from the medicinal fluid; the needle holder 3 on which the connector member 110 is fitted to be assembled/disassembled; and a needle 4 insert-molded on a tip end of the needle holder 3. In particular, the syringe is provided with a connector type filtering device including a connector member, in which the connector member is opened at both front and rear ends, an outlet and an inlet are separately formed in the connector member, respectively, for discharge and suction of the medicinal fluid, and a filter member and a packing member are inserted into the outlet and the inlet, respectively, to be assembled to or disassembled from the needle holder and the cylinder neck 1a, respectively, to be replaceable.

In order to achieve the above-described object, another embodiment of the present invention provides a connector-type filtering device for removing a foreign matter included in a medicinal fluid sucked in to the inside of a cylinder from a syringe needle or discharged from the cylinder. The filtering device includes a connector member formed as a cylindrical body which is opened at both front and rear ends to be assembled to or disassembled from the needle holder and the cylinder neck, respectively, to be replaceable, and including an outlet and an inlet which are separately formed in the cylindrical body, respectively.

In order to achieve the above-described object, still another embodiment of the present invention provides a method for manufacturing a connector-type filtering device by separately configuring connector forming molds in a lower mold and an upper mold, respectively, so that a cylindrical connector member including a holder coupling portion and a cylinder coupling portion, in which a medicinal fluid inlet and a medicinal fluid outlet are separately formed respectively, is capable of being injection-molded, and compressively pouring a resin material, which is in a fluid state by being heated, into a cavity of the connector molding molds which is formed by closing the lower mold by the upper mold to manufacture a molded product corresponding to the cavity of the connector molding molds. The method includes: a first step of mounting one or more core pins on the top of the connector forming mold of the lower mold exposed by opening the upper mold so as to fix one or more filter members at predetermined positions on the top of the connector molding mold of the lower mold; a second step of fixing the one or more filter members at the predetermined positions using the one or more core pins mounted on the top of the connector molding mold of the lower mold; and a third step of closing the upper mold on the lower mold in a state where the one or more filter members are fixed at the predetermined positions by the one or more core pins, and compressively pouring the resin material, which is in the fluid state by being heated, into the cavity of the connector molding molds which is formed by closing the upper mold so as to mold a molded filter member-inserted connector product having a shape corresponding to the cavity of the connector molding molds.

### Advantageous Effects

According to the present invention, since the filtering device is configured in the form of a connector to be compatible between the cylinder and the needle holder of a conventional ordinary syringe, the filtering device may be applied without artificially deforming the needle. Thus, in the case where it is necessary to administer a medicinal fluid by changing the needle with another needle when a blood vessel of the patient is narrow or the viscosity of the medicinal fluid is high, or in a case where it is necessary to directly administer the medicinal fluid to an injection port, the filtering device can be easily and conveniently used merely by connecting the needle holder changed to have another gauge and the cylinder or by removing the needle and the holder and directly connecting only the connector to the injection port. Thus, a product according to the present invention may secure competitiveness in the market, unlike a product in which the needle should be replaced again or in which filtering is performed at the time of discharging the medicinal fluid.

In addition, the present invention may simply apply the filtering device for each of the suction operation and discharge operation of the medicinal fluid unlike an existing product in which the filtration function is applied only one time at the time of suction or discharging the medicinal fluid. Thus, since the internal processes may be minimized and the component material may be simplified, the present invention is excellent in price competiveness, and since the dual filtration by the primary filtration at the time of sucking the medicinal fluid and the secondary filtration at the time of discharging the medicinal fluid can be performed, security at the time of injecting a medicinal fluid of an ampule or the like can be maximized.

Further, since the present invention is capable of achieving the dual filtration only with the force of pushing and pulling the syringe, an operator's convenience can be improved. In addition, replacement of the needle is not needed, the danger of secondary infection may be removed, the cost saving may be maximized, and the wastes may be halved so that the present invention may contribute to a green industry.

### BRIEF DESCRIPTION OF THE DRAWINGS

FIG. 1 is a perspective view illustrating a use example of a conventional ordinary syringe and an ampule;

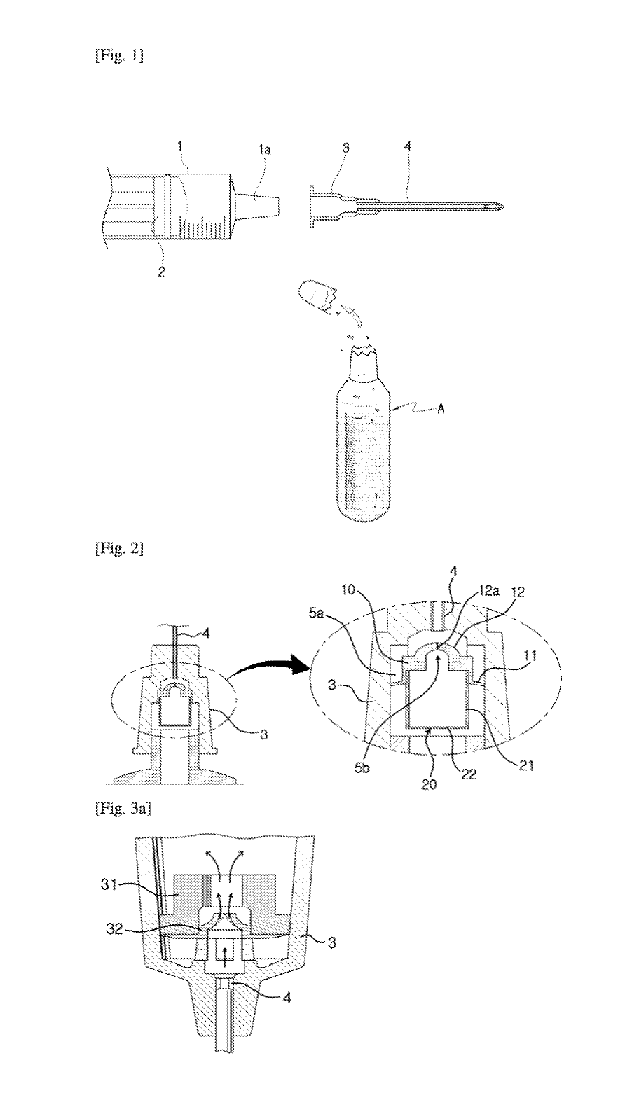

FIG. 2 is a cross-sectional view illustrating a configuration of a needle-integrated filter of the prior art;

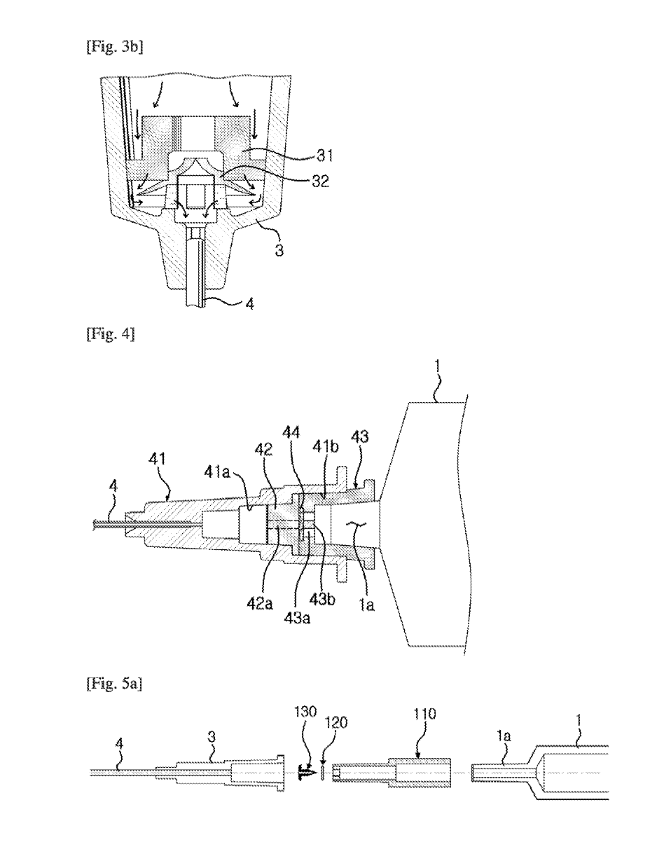

FIGS. 3a and 3b are cross-sectional views exemplifying a filter and valve assembly for a hypodermic syringe of the prior art;

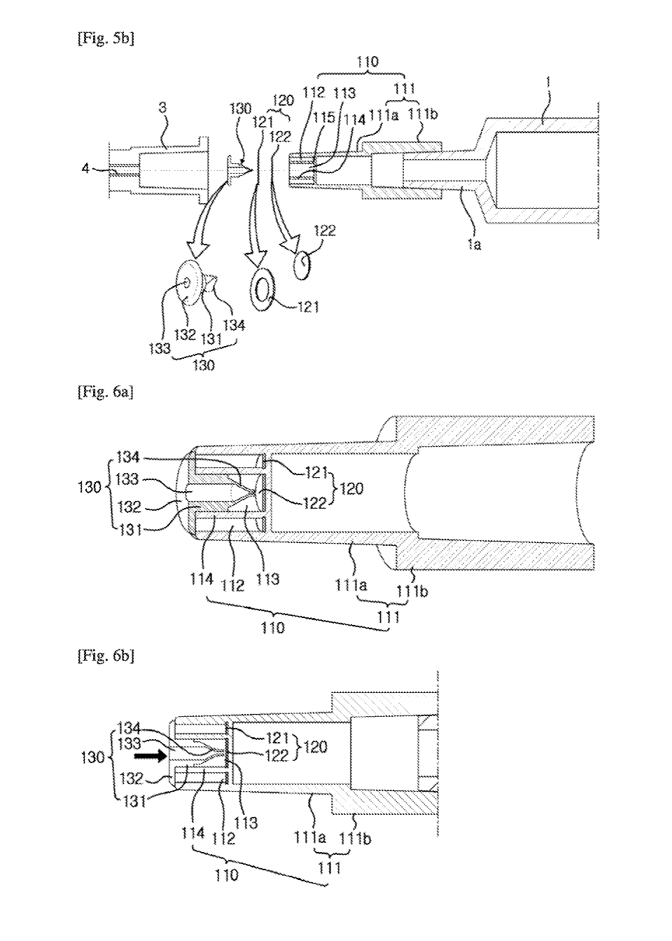

FIG. 4 is a cross-sectional view exemplifying a filter member for a syringe of the prior art;

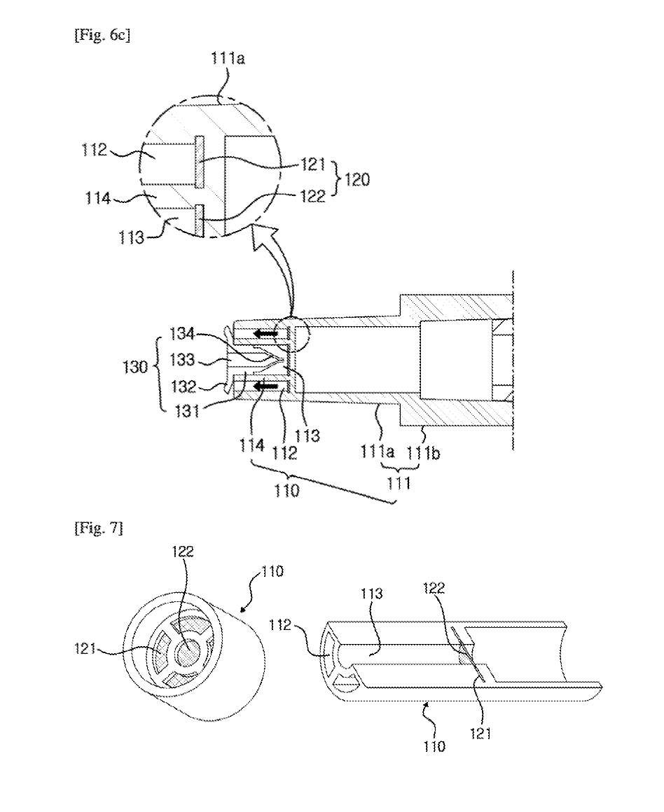

FIGS. 5a and 5b are cross-sectional views illustrating principal parts of syringes each having a connector-type filtering device according to each embodiment of the present invention in a disassembled state;

FIGS. 6a to 6c are a cut-away view exemplifying an extracted connector-type filtering device according to a second embodiment of the present invention and cross-sectional views exemplifying suction and discharge operations thereof;

FIG. 7 illustrates a bottoms side perspective view exemplifying a holder coupling portion extracted from a connector member in order to describe a filter paper insertion state in the connector filtering device according to the second embodiment of the present invention, and a perspective view exemplifying the holder coupling portion in order to exemplify a cross section thereof;

FIGS. 8a and 8b are views exemplifying a connector-type filtering device according to the present invention in a disassembled state and an assembled state in order to describe an example of applying a syringe to an injection port; and

FIGS. 9a to 9d are views exemplifying manufacturing process flow charts in order to describe a method for manufacturing a connector-type filtering device for a syringe according to the present invention.

### MODE FOR CARRYING OUT THE INVENTION

Hereinafter, configurations and overall operations of a connector-type filtering device for a syringe according to the present invention, a syringe including the same, and a method for manufacturing the connector-type filtering device will be described in detail with reference to the accompanying drawings.

Terms and words used in the specification and claims shall not be limitedly interpreted in conventional or dictionary definitions. Based on the principle that an inventor may properly define concepts of terms in order to disclose his/her invention in the best way, the terms and words shall be interpreted in the definitions and concepts to be consistent with the technical idea of the present invention. Accordingly, it shall be noted that since the embodiments described in the present specification and the configurations illustrated in the drawings are merely most preferred embodiments of the present invention, various equivalents and modified examples which may replace the embodiments and configurations may exist at the time of filing the present application.

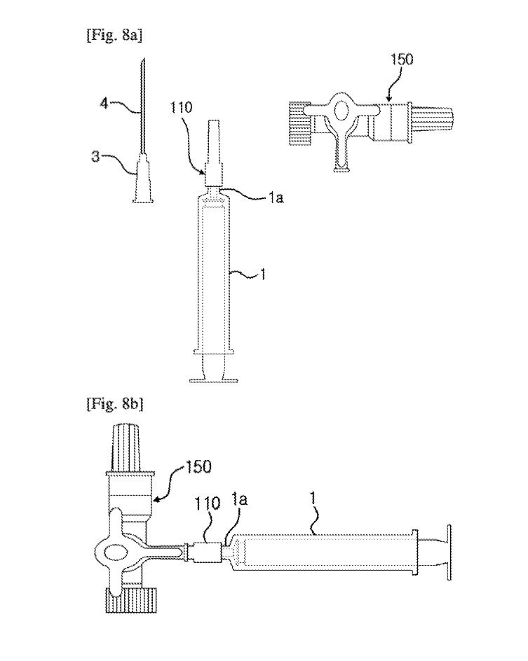

FIGS. 5a and 5b are cross-sectional views illustrating principal parts of syringes each having a connector-type filtering device according to each embodiment of the present invention in a disassembled state, in which FIGS. 5a and 5b exemplify a syringe having a connector-type filtering device provided with one filter paper and a syringe having a connector-type filtering device provided with two filter papers, respectively.

As exemplified in FIGS. 5a and 5b, a syringe having a connector-type filtering device according to each embodiment of the present invention is a syringe in which a filtering device configured to remove foreign matter included in a medicinal fluid sucked into the inside of a cylinder 1 from a needle 4 or discharged from the cylinder 1 may be assembled or disassembled. A needle holder 3 integrated with the needle 4 may be disassembled from or assembled to a cylinder neck 1a of the cylinder 1. The connector-type filtering device according to the present invention is configured to be assembled to or disassembled from a space between the needle holder 3 and the cylinder neck 1a in front of the cylinder 1. For this purpose, the connector-type filtering device includes a connector member 110 which is opened at both front and rear ends, and a filter member 120 and a packing member 130 which are inserted into or assembled to the inside of the connector member 110 to allow the medicinal fluid to flow therethrough, as exemplified in FIG. 5a.

As exemplified in FIG. 5b, the connector member 110 includes a cylindrical body 111, which is opened at both front and rear ends, in which the front portion and the rear portion of the cylindrical body 111 having different diameters to form a stepped structure. A holder coupling portion 111a is formed on the front portion to be capable of being assembled with or disassembled from the needle holder 3, and a cylinder coupling portion 111b is formed on the rear portion to be capable of being assembled with or disassembled from the cylinder neck 1a of the cylinder 1, so that both the front and rear ends of the cylindrical body 111 may be fitted to or separated from the needle holder 3 and the cylinder neck 1a, respectively.

In addition, inside the holder coupling portion 111a, an outlet 112 and an inlet 113 configured to be capable of discharging or sucking in the medicinal fluid are formed to be separated from each other by a separation wall 114 and to penetrate the holder coupling portion 111a in the axial direction, and a filter member 120 configured to filter foreign matter and a packing member 130 configured to selectively interrupt suction and discharge of the medicinal fluid are inserted into the outlet 112 and the inlet 113 to be attached and assembled, respectively.

The filter member 120 includes one or more filter papers having pores of a size limiting the passage of foreign matter contained in the medicinal fluid, and is inserted into and assembled in the inside of the holder coupling portion 111a of the connector member 110 to close the outlet 112 or the inlet 113 or both the outlet 112 and the inlet 113 of the connector member 110. At this time, the filter papers are filter papers certified by the Korean Food and Drugs Administration and having a pore size of 5.0 um or less and a height of 0.2 mm. Other filter papers may be used without any limitation as long as they may smoothly suck in or discharge a medicinal fluid.

Such a filter member 120 may be configured as a single filtration type filter member that filters foreign matter contained in the medicinal fluid discharged through the outlet 112 by being inserted and attached to cover the entire cross section of the outlet 112 inside the holder coupling portion 111a, or configured as a dual filtration type filter member that primarily filters the foreign matter of the medicinal fluid sucked in through the inlet 113 and secondarily filters the foreign matter of the medicinal fluid discharged through the outlet 112 by being inserted and attached to cover the entire cross sections of the outlet 112 and the inlet 113 inside the holder coupling portion 111a.

As illustrated in FIG. 5a, the single filtration type filter member may be preferably formed by a first filter paper 121 that is configured in a donut shape centrally opened to correspond to the cross-sectional shape of the outlet 112 inside the holder coupling portion 111a of the connector member 110, and has pores having a uniform size. In the filter member configured in this way, the foreign matter of the medicinal fluid sucked in through the inlet 113 is sucked in to the inside of the cylinder as it is without being filtered, and the foreign mater of the medicinal fluid discharged through the outlet 112 is primarily filtered and discharged by the first filter paper 121.

As illustrated in FIGS. 5a and 5b, the packing member 130 is assembled by being fitted in the inlet 113 inside the holder coupling portion 111a in front of the connector member 110 to temporarily close or open the inlet 113 of the connector member 110 by the discharge pressure of the medicinal fluid which is formed inside the holder coupling portion 111a.

For this purpose, the packing member 130 includes a cylindrical body member 131 configured to be fitted to the inside of the inlet 113 from the end at the holder coupling portion 111a side and a flange 132 configured to limit the fitting of the cylindrical body member 131, in which the cylindrical body member 131 and the flange 132 are formed integrally with each other. At the center of the cylindrical body member 131, a suction hole 133 is formed to penetrate the cylindrical body member 131 to suck in the medicinal fluid, and at the other end of the cylindrical body member 131, an elastic inclined portion 134 is formed to selectively interrupt the communication between the inlet 113 of the connector member 110 and the suction hole 133 inside the cylindrical body member 131 by the discharge pressure of the medicinal fluid which is formed inside the holder coupling portion 111a in the state where the cylindrical body member 131 is fitted in the inlet 113 of the connector member 110.

The cylindrical body member 131 is formed in a shape corresponding to the inlet 113 and having an outer diameter smaller than the inlet 113 so that the cylindrical body member 131 may be press-fitted to the inside of the inlet 113 from the end of the connector member 110 at the holder coupling portion 111a side.

The flange 132 may be formed horizontally in the circumferential direction of the cylindrical body member 131 at the rear end of the cylindrical body member 131 and integrally with the cylindrical body member 131 so as to limit the fitting of the cylindrical body member 131 to the inlet 113 of the holder coupling portion 111a at one end of the cylindrical body member 131 (e.g., at the rear end of the cylindrical body member 131). In particular, the flange 132 is formed to have a width that may entirely cover the outlet 112 of the holder coupling portion 111a. The flange 132 is preferably formed of a material having a thickness and elasticity that is capable of sufficiently performing the following operations: in a normal condition, the peripheral edge of the flange 132 may entirely cover the outlet 112 of the holder coupling portion 111a, and when the medicinal fluid is sucked in to the outlet 112 through the filter member 120 by the discharge pressure of the medicinal fluid which is formed inside the holder coupling portion 111a, the peripheral edge of the flange 132 is lifted by the medicinal fluid discharge pressure of the outlet 112 to open the outlet 112 such that the medicinal fluid may pass through the outlet 112 to be easily discharged to the needle holder 3 (or the injection port) side.

The suction hole 133 is formed at the center of the cylindrical body member 131 so as to suck in the medicinal fluid and formed to penetrate the cylindrical body member 131 in the axial direction.

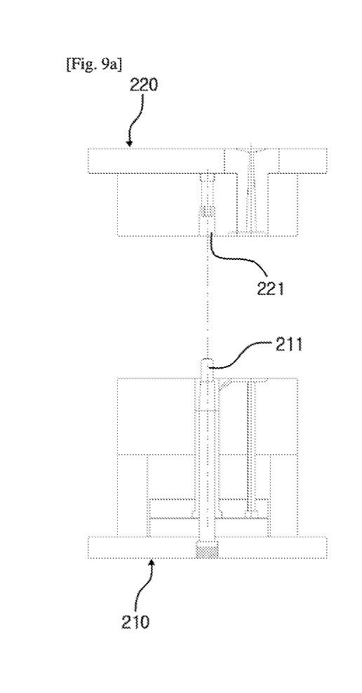

The elastic inclined portion 134 is formed on the other end of the cylindrical body member 131 opposite to the end where the flange 132 is formed. The elastic inclined portion 134 is made of a material having a thickness and elasticity sufficiently allowing the peripheral edge of the elastic inclined portion 134 to be elastically closed by the medicinal fluid discharge pressure formed inside the holder coupling portion 111a in the state where it is fitted in the inlet 113 of the connector member 110 (see FIG. 6c) and to allow the peripheral edge to be opened by the suction pressure of the medicinal fluid flowing into the suction hole 133 from the outside (see FIG. 6b). The elastic inclined portion 134 functions to selectively adjust the communication between the inlet 113 of the connector member 110 and the suction hole 133 inside the cylindrical body member 131.

FIGS. 6a to 6c are a cut-away view exemplifying an extracted connector-type filtering device according to a second embodiment of the present invention and cross-sectional views exemplifying suction and discharge operations thereof, in which FIG. 6a is a cut-away perspective view of the connector-type filtering device, into which a dual filtration type filter member is inserted, FIG. 6b is a cross-sectional view exemplifying a medicinal fluid suction operation in the connector-type filtering device, into which the dual filtration type filter member is inserted, and FIG. 6c is a cross-sectional view exemplifying a discharge operation in the connector-type filtering device, into which the dual filtration type filter member is inserted.

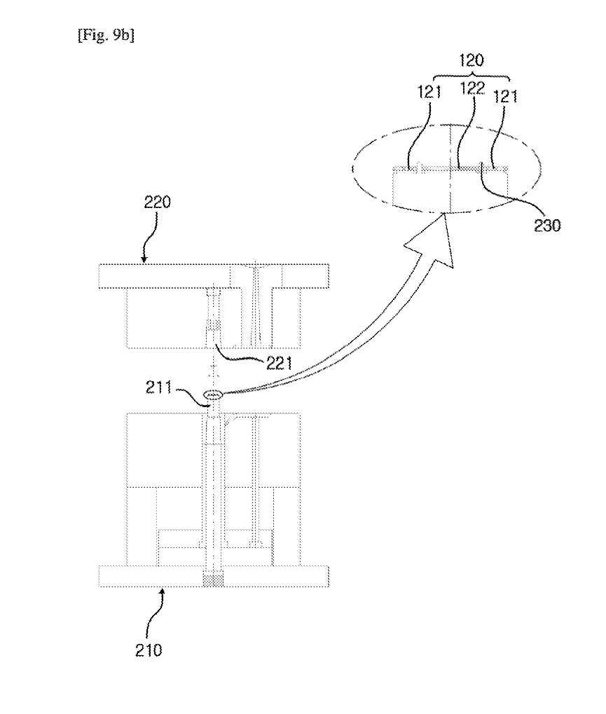

FIG. 7 illustrates a bottom side perspective view exemplifying a holder coupling portion extracted from a connector member in order to describe a filter paper insertion state in the connector filtering device according to the second embodiment of the present invention, and a perspective view exemplifying the holder coupling portion in order to exemplify a cross section thereof, in which a state, in which the dual filtration type filter member is inserted into and assembled to the inside of the holder coupling portion 111a of the connector member 110, is exemplified.

As exemplified in FIG. 5b and FIGS. 6a to 6c, the dual filtration type filter member may include: a first filter paper 121 formed in a donut shape, of which a center is opened to correspond to the cross-sectional shape of the outlet 112 of the connector member 110, and having pores of a uniform size; and a second filter paper 122 formed in a circular shape corresponding to a cross-sectional shape of the inlet 113 of the connector member 110, and having pores of a size which is different from that of the first filter paper 121. In the filter member configured as described above, the foreign matter of the medicinal fluid sucked in through the inlet 113 is primarily filtered by the second filter paper 122 and sucked in to the inside of the cylinder, and the foreign matter of the medicinal fluid discharged through the outlet 112 is secondarily filtered by the first filter paper 121 and discharged, as exemplified in FIG. 6b.

Accordingly, in order to cope with various medical environments, the single filtration type using only the first filter paper or the second filter paper, and the dual filtration type using both the first filter paper and the second filter paper may be selectively implemented.

Still another dual filtration type filter member may also be configured by a third filter paper (not exemplified in the drawing) which has pores having a uniform size and is formed in a circular shape to correspond to a cross-sectional shape obtained by combining the outlet 112 and the inlet 113 so as to entirely cover both the outlet 112 and the inlet 113 inside the holder coupling portion 111a of the connector member 110. In the filter member configured in this way, the foreign matter of the medicinal fluid sucked in through the inlet 113 is primarily filtered in the central region of the third filter paper and sucked in to the inside of the cylinder, and the foreign matter of the medicinal fluid discharged through the outlet 112 is secondarily filtered in the donut-shaped region of the third filter paper and discharged.

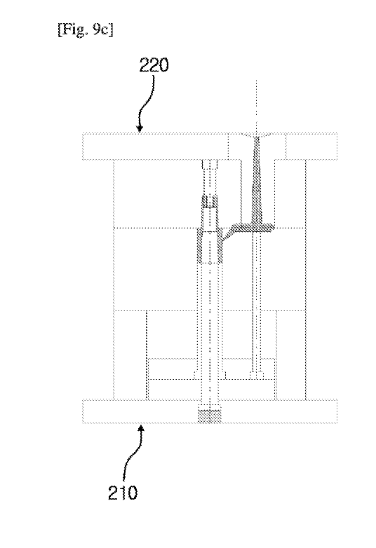

FIGS. 8a and 8b are views exemplifying a connector-type filtering device according to the present invention in a disassembled state and an assembled state in order to describe an example of applying a syringe to an injection port. In particular, FIGS. 8a and 8b illustrate a state in which, in the configuration of the syringe in which the connector-type filtering device mounted on the cylinder neck 1a of the cylinder 1, the needle holder 3, to which the needle 4 is integrally fixed, is removed from the connector member 110 of the connector-type filtering device and then the connector member 110 of the connector-type filtering device is directly connected to the injection port 150.

As exemplified in FIGS. 8a and 8b, when it is necessary to change the needle to another needle of a different gauge so as to administer the medicinal fluid, or to directly administer the medicinal fluid to the injection port as a blood vessel of a patient is narrow or the viscosity of the medicinal fluid is high, the syringe having the connector-type filtering device according to the present invention may be used as a syringe of the other gauge merely by connecting the connector between the needle holder changed to the other gauge and the cylinder or may be easily used by removing the needle and the holder and directly connecting only the connector member 110 of the connector-type filtering device to the injection port, as exemplified in FIG. 8b.

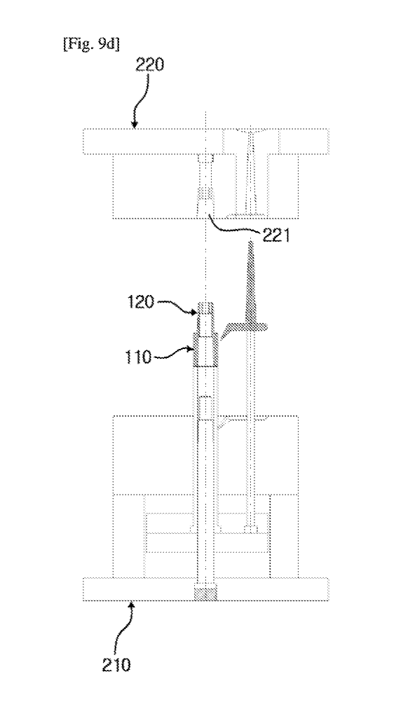

FIGS. 9a to 9d are views exemplifying manufacturing process flow charts in order to describe a method for manufacturing a connector-type filtering device for a syringe according to the present invention. As exemplified in FIGS. 5a and 5b and FIGS. 6a to 6c, the cylindrical connector member 110 configured by the holder coupling portion 111a and the cylinder coupling portion 111b which respectively form the medicinal fluid inlet 113 and the medicinal fluid outlet 112 therein to be separated from each other should be molded by injection molding. Thus, as exemplified in FIG. 9a, an injection molding apparatus used for performing the manufacturing method of the present invention is implemented such that connector molding molds 211 and 221 are separately formed in a lower mold 210 and an upper mold 220, respectively, and a resin material heated to be in a fluid state is compressively poured into a cavity of the connector molding molds, which is formed by closing the lower mold 210 with the upper mold 220, and then is cooled within the connector molding molds so that a molded product corresponding to the cavity of the connector molding molds can be manufactured.

The connector-type filtering device manufacturing method of the present invention performed in the molded product manufacturing apparatus implemented as described above is generally divided into first to third steps.

As exemplified in FIG. 9a, the first step (a) is a step for mounting one or more core pins 230 for fixing one or more filter members 120 at predetermined positions on the top of the connector forming mold 211 of the lower mold 210 exposed in a state where the lower mold 210 is exposed by opening the upper mold 220. At this time, the core pins 230 are mounted such that protrusions thereof protrude to the upper mold 220 side. In addition, the core pins 230 are mounted to be arranged circumferentially along the periphery of the medicinal fluid inlet 113 to be formed inside of the holder coupling portion 111a of the connector member 110 molded by the connector molding molds.

As exemplified in FIG. 9b, the second step (b1) is a step for fixing the one or more filter members 121 at predetermined positions using the core pins 230 mounted on the top of the connector molding mold 211 of the lower mold 210. The second step (b) may be divided into: step (b1) of fixedly attaching an inner periphery of a first filter paper 121, which is opened in a donut shape to correspond to the cross-sectional shape of the medicinal fluid outlet 112 and has pores of a uniform size, to the inside of the holder coupling portion 111a of the connector molding mold by the one or more core pins 230; and step (b2) of fixing a second filter paper 122, which is formed in a circular shape to correspond to a cross-sectional shape of the medicinal fluid inlet 113 formed inside the holder coupling portion 111a in the connector molding molds and has pores having a size which is equal to or different from that of pores of the first filter paper 121, by the core pins 230. Of course, the first filter paper fixing step b1 and the second filter paper fixing step b2 may be performed in the reversed order.

In addition, the step of inserting and fixing the filter paper to the mold may be performed manually or by using an automated apparatus.

At this time, in the first filter paper fixing step (b1), the first filter paper may be attached by fixedly attach the inner periphery opened in the donut shape of the first filter paper 121 to the inside of the holder coupling portion 111a of the connector molding mold by the one or more core pins 230, and by inserting and fixing the outer periphery of the first filter paper 121 to an insertion groove 115 formed along the inner circumferential surface of the cylindrical body 111 of the connector member 110.

In addition, in the second filter paper fixing step (b2), the second filter paper may be attached by fixing the circular periphery of the second filter paper 122 to the inside of the holder coupling portion 111a of the connector molding mold by the one or more core pins 230.

The third step (c) is a step of closing the upper mold 220 on the lower mold 210 as illustrated in FIG. 9c, in a state where the one or more filter members 120 are fixed at the predetermined positions by the one or more core pins 230, compressively pouring the resin material heated to be in the fluid state into the cavity of the connector molding molds, which is formed by closing the upper mold 220, and cooling the resin material within the connector molding molds, thereby manufacturing a molded filter member-inserted connector product having a shape corresponding to the cavity of the connector molding molds. After these procedures, as exemplified in FIG. 9d, when the upper mold is opened to take out the molded connector product, a connector-type filtering device of a single filtration type or a connector-type filtering device of a dual filtration type, which is formed by finally inserting one or two filters, may be manufactured through injection molding.

As described above, the present invention mounts a connector-type filtering device as an intermediate medium which may be interposed between a needle holder and a cylinder of a syringe to be compatible with a conventional syringe so that, when it is necessary to change the needle to another needle of a different gauge so as to administer the medicinal fluid or to directly administer the medicinal fluid to the injection port as a blood vessel of a patient is narrow or the viscosity of the medicinal fluid is high, the syringe may be easily used merely by connecting the connector between the needle holder changed to the other gauge and the cylinder or by removing the needle and the holder and directly connecting only the connector member 110 of the connector-type filtering device to the injection port. In particular, the present invention applies a filtering device for each of medicinal fluid suction and discharge operations, so that dual filtration may be performed by primary filtration at the time of sucking in the medicinal fluid and secondary filtration at the time of discharging the medicinal fluid, thereby maximizing safety at the time of injecting a medicinal fluid of an ampule or the like.

In the foregoing, although the present invention has been described with reference to limited embodiments and drawings, the present invention is not limited to the embodiments described above and a person skilled in the art to which the present invention belongs may made various changes and modifications from the above descriptions. Accordingly, the technical idea of the present invention should be determined only based on the accompanying claims and all equivalents or equivalent modifications belong to the scope of the present invention.

### INDUSTRIAL APPLICABILITY

The present invention configures a filtering device in the form of a connector to be compatible between a cylinder and a needle holder of a conventional ordinary syringe so that a medicinal fluid, from which foreign matter is safely removed, can be administered to a patient. Thus, the present invention may be available in the medical industry in which improvement of safety is desired.

In addition, since the filtering device of the present invention can be easily and conveniently used merely by connecting the connector between the needle holder and the cylinder, or by removing the needle holder and directly connecting only the connector to an injection port. Thus, a product itself may secure competitiveness unlike a product in which the needle should be replaced again or in which filtering is performed at the time of discharging the medicinal fluid, and thus the industrial applicability of the present invention is high.

1. A syringe with a connector-type filtering device for use in administering a medicinal fluid to a patient, the syringe comprising:
a cylinder 1 having a space formed to store the medicinal fluid therein;
a piston 2 configured to reciprocate within the space of the cylinder 1 to suck in or discharge the medicinal fluid;
a connector member 110 assembled/dissembled between a needle holder 3 and a neck 1a in front of the cylinder and having a function for removing foreign matter from the medicinal fluid; the needle holder 3 configured such that the connector member 110 is fitted in or disassembled from the needle holder 3; and
a needle 4 insert-molded on a tip end of the needle holder 3.

2. The syringe of claim 1, wherein the connector member 110 is opened at both front and rear ends, an outlet 112 and an inlet 113 are separately formed in the connector member 110 to discharge and intake the medicinal fluid, and a filter member 120 and a packing member 130 are individually inserted into an entire passage route of the medicinal fluid to be assembled to or disassembled from the needle holder 3 and the cylinder neck 1a, respectively, to be replaceable.

3. The syringe of claim 1, wherein the connector member 110 forms the holder coupling portion 111a and a cylinder coupling portion 111b, which are respectively fitted to the needle holder 3 and the cylinder neck 1a to be separable, by being opened at both front and rear ends of a cylindrical body 111,
wherein the outlet 112 and the inlet 113 configured to discharge and suck in the medicinal fluid are formed in the holder coupling portion 111a to penetrate the holder coupling portion 111a in an axial direction and to be separated from each other by a separation wall 114, and
wherein the filter member 120 configured to filter the foreign matter and the packing member 130 configured to selectively interrupt suction and discharge of the medicinal fluid are respectively inserted into and attached to the outlet 112 and the inlet 113 to be assembled.

4. The syringe of claim 2, wherein the filter member 120 is configured as a single filtration type filter member inserted and attached to cover an entire cross section of the outlet 112 inside a colder coupling portion 111a so as to filter the foreign matter contained in the medicinal fluid discharged through the outlet 112.

5. The syringe of claim 4, wherein the single filtration type filter member is made of a first filter paper 121 of which the central portion is opened in a donut shape to correspond to the cross-sectional shape of the outlet 112 and having pores of a uniform size.

6. The syringe of claim 2, wherein the filter member 120 is configured as a dual filtration type filter member inserted and attached to cover an entire cross section of the outlet 112 and the inlet 113 inside a holder coupling portion 111a so as to primarily filter the foreign matter in the medicinal fluid sucked in through the inlet 113 and so as to secondarily filter the foreign matter from the medicinal fluid discharged through the outlet 112.

7. The syringe of claim 6, wherein the dual filtration type filter member is made of a first filter paper 121 which is inserted and attached to cover the entire cross section of the outlet 112 and the inlet 113 inside the holder coupling portion 111a and has pores having a uniform size.

8. The syringe of claim 6, wherein the dual filtration type filter member includes:
a first filter paper 121 formed in a donut shape, of which a center is opened to correspond to the cross-sectional shape of the outlet 112 of the connector member 110, and having pores of a uniform size; and
a second filter paper 122 formed in a circular shape corresponding to a cross-sectional shape of the inlet 113 of the connector member 110, and having pores, a size of which is different from that of the first filter paper 121.

9. The syringe of claim 1, wherein, in the connector member 110, the inlet 113 configured to suck the medicinal fluid is formed in the central portion of the cylindrical body 111 to penetrate the cylindrical body in an axial direction, and the outlet 112 configured to discharge the medicinal fluid is formed around the inlet 113 to penetrate the cylindrical body 111 in the axial direction,
wherein a first filter paper 121 is formed in a donut shape, of which a center is opened to correspond to the cross-sectional shape of the outlet 112 of the connector member 110, and inserted into and assembled to the inside of the holder coupling portion 111a so as to block the outlet 112 of the connector member 110, and
wherein a second filter paper 122 is formed in a circular shape corresponding to a cross-sectional shape of the inlet 113 of the connector member 110, and inserted into and assembled to the inside of the holder coupling portion 111a of the connector member 110 so as to block the inlet 113 of the connector member 110.

10. The syringe of claim 2, wherein packing member 130 includes:
a cylindrical body member 131 configured to be fitted into the inlet 113 from an end of the connector member 110 at the holder coupling portion 111a side; and
a flange 132 integrally formed on the cylindrical body member 131 so as to limit the fitting of the cylindrical body at one end of the cylindrical body member 131.

11. The syringe of claim 10, wherein the packing member 130 includes a suction hole 133 formed in a central portion of the cylindrical body member 131 to penetrate the cylindrical body member 131 in an axial direction so as to suck in the medicinal fluid, and
wherein the cylindrical body member 131 includes an elastic inclined portion 134 formed on the other end of the cylindrical body member 131 so as to selectively interrupt communication between the inlet 113 of the connector member 110 and the suction hole 133 inside the cylindrical body member 131 by a discharge pressure of the medicinal fluid formed inside the holder coupling portion 111a in a state where the cylindrical body member 131 is fitted to the inlet 113 of the connector member 110.

12. A connector-type filtering device for removing a foreign matter included in a medicinal fluid sucked into a cylinder 1 from a syringe needle 4 or discharged from the cylinder 1, the filtering device comprising:
a connector member 110 made of a cylindrical body 111, which is opened at both front and rear ends, and including an outlet 112 and an inlet 113, which are separately formed in the cylindrical body 111, a filter member 120, and a packing member 130, the filter member 120 and the packing member 130 being individually inserted into an entire passage route of the medicinal fluid,
wherein the filter member 120 includes a dual filtration type filter member inserted and attached to cover an entire cross section of the outlet 112 and the inlet 113 inside a holder coupling portion 111a so as to primarily filter the foreign matter in the medicinal fluid sucked in through the inlet 113 and to secondarily filter the foreign matter from the medicinal fluid discharged through the outlet 112.

13. The connector-type filtering device of claim 12, wherein the connector member 110 forms the holder coupling portion 111a and a cylinder coupling portion 111b, which are respectively fitted to the needle holder and the cylinder neck 1a, by being opened at both front and rear ends of a cylindrical body 111,
wherein the outlet 112 and the inlet 113 configured to discharge and suck in the medicinal fluid are formed in the holder coupling portion 111a to penetrate the holder coupling portion 111a in an axial direction and to be separated from each other by a separation wall 114, and
wherein the filter member 120 configured to filter the foreign matter and the packing member 130 configured to selectively interrupt suction and discharge of the medicinal fluid are respectively inserted into the outlet 112 and the inlet 113 to be assembled.

14. The syringe of claim 12, wherein the filter member 120 is configured as a single filtration type filter member inserted and attached to cover an entire cross section of the outlet 112 inside a colder coupling portion 111a so as to filter the foreign matter contained in the medicinal fluid discharged through the outlet 112.

15. The syringe of claim 14, wherein the single filtration type filter member is made of a first filter paper 121 of which the central portion is opened in a donut shape to correspond to the cross-sectional shape of the outlet 112 and having pores of a uniform size.

16. The syringe of claim 12, wherein the filter member 120 is configured as a dual filtration type filter member inserted and attached to cover an entire cross section of the outlet 112 and the inlet 113 inside a holder coupling portion 111a so as to primarily filter the foreign matter in the medicinal fluid sucked in through the inlet 113 and so as to secondarily filter the foreign matter from the medicinal fluid discharged through the outlet 112.

17. The connector-type filtering device of claim 16, wherein the dual filtration type filter member is made of a first filter paper 121 which is inserted and attached to cover the entire cross section of the outlet 112 and the inlet 113 inside the holder coupling portion 111a and has pores having a uniform size.

18. The connector-type filtering device of claim 16, wherein the dual filtration type filter member includes:
a first filter paper formed in a donut shape, of which a center is opened to correspond to the cross-sectional shape of the outlet 112 of the connector member 110, and having pores of a uniform size; and
a second filter paper 122 formed in a circular shape corresponding to a cross-sectional shape of the inlet 113 of the connector member 110, and having pores of a size which is equal to or different from that of the first filter paper 121.

19. The connector-type filtering device of claim 12, wherein, in the connector member 110, the inlet 113 configured to suck the medicinal fluid is formed in the central portion of the cylindrical body 111 to penetrate the cylindrical body in an axial direction, and the outlet 112 configured to discharge the medicinal fluid is formed around the inlet 113 to penetrate the cylindrical body 111 in the axial direction,
a filter paper 121 is formed in a donut shape, of which a center is opened to correspond to the cross-sectional shape of the outlet 112 of the connector member 110, and inserted into and assembled to the inside of the holder coupling portion 111a so as to block the outlet 112 of the connector member 110,
a second filter paper 122 is formed in a circular shape corresponding to a cross-sectional shape of the inlet 113 of the connector member 110, and inserted into and assembled to the inside of the holder coupling portion 111a of the connector member 110 so as to block the inlet 113 of the connector member 110.

20. The connector-type filtering device of claim 12, wherein packing member 130 includes:
a cylindrical body member 131 configured to be fitted into the inlet 113 from an end of the connector member 110 at the holder coupling portion 111a side; and
a flange 132 integrally formed on the cylindrical body member 131 so as to limit the fitting of the cylindrical body at one end of the cylindrical body member 131.

21. The connector-type filtering device of claim 20, wherein the packing member 130 includes a suction hole 133 formed in a central portion of the cylindrical body member 131 to penetrate the cylindrical body member 131 in an axial direction so as to suck in the medicinal fluid, and
wherein, the cylindrical body member 131 includes an elastic inclined portion 134 formed on the other end of the cylindrical body member 131 so as to selectively interrupt communication between the inlet 113 of the connector member 110 and the suction hole 133 inside the cylindrical body member 131 by a discharge pressure of the medicinal fluid formed inside the holder coupling portion 111a in a state where the cylindrical body member 131 is fitted to the inlet 113 of the connector member 110.

22. A method for manufacturing a connector-type filtering device by separately configuring connector forming molds 211 and 221 in a lower mold 210 and an upper mold 220, respectively, and compressively pouring a resin material, which is in a fluid state by being heated, into a cavity of the connector molding molds 211 and 221 which is formed by closing the lower mold 210 by the upper mold 220, the method comprising:
(a) a first step of mounting one or more core pins 230 for fixing one or more filter members 120 at predetermined positions on the top of the connector forming mold 211 of the lower mold 210 exposed in a state where the lower mold 230 is exposed by opening the upper mold 220;
(b) a second step of fixing the one or more filter members 120 at the predetermined positions using the one or more core pins 230 mounted on the top of the connector molding mold 211 of the lower mold 210; and
(c) a third step of closing the upper mold 220 on the lower mold 210 in a state where the one or more filter members 120 are fixed at the predetermined position by the one or more core pins 230, and compressively pouring the resin material in the fluid state into the cavity of the connector molding molds which is formed by closing the upper mold 220 so as to mold a filter member-inserted connector member having a shape corresponding to the cavity of the connector molding molds.

23. The method of claim 22, wherein, in the first step, the core pins 230 are mounted to protrude toward the upper mold.

24. The method of claim 22, wherein, in the first step, the one or more core pins 230 are mounted to be arranged circumferentially along a periphery of the inlet 113 for sucking the medicinal fluid which is to be formed inside the holder coupling portion 111a of the connector member 110 which is to be formed by the connector molding molds.

25. The method of claim 22, wherein the second step includes:
(b1) fixing a first filter paper 121, of which the central portion is opened in a donut shape to correspond to the cross-sectional shape of the outlet 112 and having pores of a uniform size, by the core pins 230.

26. The method of claim 25, wherein the second step includes:
a step of fixedly attaching an inner periphery of the first filter paper 121 opened in a donut shape to an inside of the holder coupling portion 111a in the connector molding mold by the one or more core pins 230, and attaching an outer periphery of the first filter paper 121 by inserting and fixing the outer periphery of the first filter paper 121 in an insertion groove 115 formed along an inner circumferential surface of a cylindrical body 111 of a connector member 110.

27. The method of claim 25, wherein the second step further includes:
(b2) a step of fixing a second filter paper 122 formed in a circular shape corresponding to a cross-sectional shape of an inlet 113 for sucking in a medicinal fluid which is formed inside the holder coupling portion 111a in the connector molding mold, the second filter paper 122 having pores having a size which is different from that of pores of the first filter paper 121.

28. The method of claim 27, wherein, in the second step, the circular periphery of the second filter paper 122 is fixedly attached to the inside of the holder coupling portion 111a of the connector molding mold by the one or more core pins 230.
